(19) United States
(12) Patent Application Publication
(10) Pub. No.: US 2016/0166700 A1
(43) Pub. Date: Jun. 16, 2016

(54) METHOD FOR AMELIORATING LIVER FIBROSIS BY USING NANOPARTICLE CONTAINING TYROSINE KINASE INHIBITOR

(71) Applicant: National Tsing Hua University, Hsinchu (TW)

(72) Inventors: Yun-Ching Chen, Hsinchu (TW); Ts-Ting Lin, Hsinchu (TW); Dong-Yu Gao, Hsinchu (TW)

(21) Appl. No.: 14/635,801

(22) Filed: Mar. 2, 2015

(30) Foreign Application Priority Data
Dec. 11, 2014 (TW) 103143303

(51) Int. Cl. A61K 47/34; A61K 31/428; A61K 9/00; A61K 31/44

(52) U.S. Cl. CPC A61K 47/34; A61K 31/44; A61K 31/428; A61K 9/0019

(57) ABSTRACT

The present invention is related to a method for ameliorating liver fibrosis in a subject. The present invention utilizes copolymers of polyethylene glycol and poly (D, L-lactide-co-glycolide) to form nanoparticle composition containing tyrosine kinase inhibitor, such as Sorafenib. The use of nanoparticle composition is non-toxic and can increase the stability and decrease the release of drug in blood circulation. The pharmaceutical composition is in an injectable form. The nanoparticle composition containing tyrosine kinase inhibitor of the present invention can effectively ameliorate liver fibrosis including decreasing extracellular matrix accumulation, suppressing hepatic stellate cell activity, shrinking of abnormal blood vessel, and lowering microvascular density in fibrotic liver; hence is suitable for clinical application for the treatment of liver fibrosis.

### CROSS-REFERENCES TO RELATED APPLICATIONS

This application claims priority to Taiwan Patent Application No. 103143303 filed on 11 Dec. 2014. All disclosure of which is incorporated herein by reference in its entirety.

### BACKGROUND OF THE INVENTION

1. Field of the Invention

The present invention is related to a method for ameliorating liver fibrosis. Particularly, the present invention is related to a method for ameliorating liver fibrosis by using a nanoparticle composition.

2. the Prior Arts

Liver fibrosis is a scar formation process resulting from chronic liver injuries and would lead to cirrhosis, liver failure, and ultimately, liver cancer. Such chronic liver injuries include: alcoholism, nonalcoholic steatohepatitis (NASH), persistent viral or helminthic infections (specifically hepatitis B and C virus infection), and hereditary metal overload, etc. The occurrence and progression of liver fibrosis is often slow with incubation period from 3 to 5 years or even up to 10 years. The progression of liver fibrosis involves angiogenesis and the generation and accumulation of extracellular matrix (ECM). Hepatic stellate cells (HSCs) are the major effector cells for regulating the fibrotic progression. Chronic liver injuries lead to production of cytokines and mitogens such as platelet-derived growth factor (PDGF) and transforming growth factor-β (TGF-β), which induce the differentiation and activation of hepatic stellate cells. The activated hepatic stellate cells then promote extracellular matrix synthesis and deposition in the fibrotic liver, which induce further release of cytokines that promote inflammation, proliferation, and angiogenesis including PDGF and vascular endothelial growth factor (VEGF). Thus, hepatic stellate cells are regarded as primary therapeutic targets for liver fibrosis or cirrhosis.

Tyrosine kinases catalyze the transfer of γ-phosphate from adenosine triphosphate (ATP) to tyrosine residue in protein and play an important role in cell growth, proliferation, and differentiation. In normal cells, the activities of tyrosine kinases are controlled under strict physiological regulations. On the other hand, in abnormal cells such as fibrotic cells or cancer cells, the over-expression or mutation of tyrosine kinases receptors leads to continuous differentiation, proliferation, anti-apoptosis, angiogenesis, and migration of cells. Thus, inhibition of the tyrosine kinase activity can prevent the tyrosine kinase receptors from over-expression and resume control of those abnormal cells. Sorafenib is a dual-function tyrosine kinase inhibitor blocking Raf/MEK/ERK pathway and vascular endothelial growth factor receptor (VEGFR)/platelet-derived growth factor receptor (PDGFR) that is used as an anti-cancer drug for attenuating angiogenesis and suppressing tumor progression. Recently, researches have shown that sorafenib can inhibit proliferation and trigger apoptosis via downregulation of cyclin Dl and cyclin-dependent kinase 4 (Cdk-4), overexpression of Fas and Fas-L and activation of caspase-3 in hepatic stellate cells. Furthermore, sorafenib also reduces the activation and differentiation of hepatic stellate cells by targeting the Raf/MEK/ERK pathway. In the experimental model using fibrotic liver, sorafenib treatment attenuates collagen deposition, reduces activation of hepatic stellate cells, and inhibits angiogenesis in the fibrotic liver, hence, resulting in amelioration of liver fibrosis.

Although sorafenib has been shown to be a potential therapeutic agent for liver fibrosis and cirrhosis, it causes side effects such as hand-foot syndrome, diarrhea, and hypertension, due to oral administration and its non-specific uptake by normal tissues. Besides, the poor water solubility of sorafenib reduces the efficiency of its absorption by the gastrointestinal tract, resulting in poor pharmacokinetics. Consequently, conventional oral administration of sorafenib for the treatment of liver fibrosis or cirrhosis in clinical applications still suffers from high recurrence rate. Taken together, the high unwanted toxicity and low bioavailability of sorafenib result in a narrow therapeutic window. Thus, a strategy to improve the absorption in the target tissues and decrease the side effects of sorafenib is of urgent necessity in order to achieve better therapeutic effects.

### SUMMARY OF THE INVENTION

As a result, the present invention provides a method for ameliorating liver fibrosis in a subject, comprising administering to the subject a nanoparticle composition comprising a tyrosine kinase inhibitor, wherein the nanoparticle composition enhances the absorption of the tyrosine kinase inhibitor in fibrotic liver and delays the release of the tyrosine kinase inhibitor in blood circulation. The pharmaceutical composition is in an injectable form.

In one embodiment of the present invention, the tyrosine kinase inhibitor is sorafenib; the nanoparticle composition reduces the accumulation of extracellular matrix in fibrotic liver. The extracellular matrix is collagen. Meanwhile, the nanoparticle composition also inhibits the activity of hepatic stellate cells.

In another embodiment of the present invention, the nanoparticle composition reduces the angiogenesis in fibrotic liver and reduces the density and diameter of newly developed blood vessels during the angiogenesis in fibrotic liver.

According to the method for ameliorating liver fibrosis in a subject of the present invention, such nanoparticle composition is a polymer of poly(ethylene glycol-b-(D, L-lactide-co-glycolide) (PEG-PLGA), or a co-polymer of poly(ethylene glycol-b-(D, L-lactide-co-glycolide)/poly(D, L-lactide-co-glycolide (PEG-PLGA/PLGA); the content of PEG-PLGA in the co-polymer of PEG-PLGAA/PLGA is from 50% to 70% by weight, and preferably, the content of PEG-PLGA and the content of PLGA in the co-polymer of PEG-PLGA/PLGA are both 50% by weight.

The low dispersion of the nanoparticle composition containing tyrosine kinase inhibitor of the present invention results in stabilized circulation in blood and delayed release of tyrosine kinase inhibitor. Furthermore, according to the present invention, the use of PEG-PLGA polymer or PEG-PLGA/PLGA co-polymer to form nanoparticle containing tyrosine kinase inhibitor, such as sorafenib, can improve or ameliorate liver fibrosis, by, for example, reducing the accumulation of extracellular matrix in fibrotic liver, suppressing the activity of hepatic stellate cells, and reducing the density and diameter of newly developed blood vessels during the angiogenesis in fibrotic liver. The PEG-PLGA polymer and PEG-PLGA/PLGA co-polymer of the present invention are non-cytotoxic and can prevent side effects which provide a safer and more efficient use of tyrosine kinase inhibitor to be applied in clinical treatment of liver fibrosis.

The present invention is further explained in the following embodiments, illustrations, and examples. Those examples below should not, however, be considered to limit the scope of the invention, and it is contemplated that modifications will readily occur to those skilled in the art, which modifications will be within the spirit of the invention and scope of the appended claims.

### BRIEF DESCRIPTION OF THE DRAWINGS

FIG. 1A, the size of the nanoparticle composition of the present invention in different solvents.

FIG. 1B, the size of the nanoparticle composition of the present invention in solvents with different ratios of ethanol to tetrahydrofuran (v/v).

FIG. 1C, the size of the nanoparticle composition of the present invention formed with different ratios of sorafenib to PEG-PLGA polymer (wt/wt).

FIG. 2A, the size of the nanoparticle composition of the present invention with different ratios of PEG-PLGA to PLGA.

FIG. 2B, the zeta-potential of the nanoparticle composition of the present invention with different ratios of PEG-PLGA to PLGA.

FIG. 2C, the encapsulation efficiency of the nanoparticle composition of the present invention with different ratios of PEG-PLGA to PLGA.

FIG. 3, electron microscopic image of nanoparticle composition containing sorafenib of the present invention consisting of PEG-PLGA polymer, and PEG-PLGA/PLGA co-polymer. The scale represents 100 nm.

FIG. 4, cumulative release rate of the sorafenib-loaded PEG-PLGA nanoparticle composition and the sorafenib-loaded PEG-PLGA/PLGA nanoparticle composition of the present invention.

FIG. 5A, cytotoxicity of the sorafenib-loaded nanoparticle composition of the present invention in human umbilical vein endothelial cells.

FIG. 5B, cytotoxicity of the sorafenib-loaded nanoparticle composition of the present invention in hepatic stellate cells.

FIG. 6, pharmacokinetics of the sorafenib-loaded nanoparticle composition of the present invention.

FIG. 7A, accumulation of the sorafenib-loaded nanoparticle composition of the present invention in fibrotic liver.

FIG. 7B, accumulation of the sorafenib-loaded nanoparticle composition of the present invention in normal liver.

FIG. 7C, fluorescent microscopic image of the cumulative effect of the sorafenib-loaded nanoparticle composition of the present invention in fibrotic and normal liver.

FIG. 8, the effect of suppressing liver fibrosis of the sorafenib-loaded nanoparticle composition of the present invention.

FIG. 9A, western blotting of the expressions of α-SMA and β-actin.

FIG. 9B, the effect of reducing extracellular matrix of the sorafenib-loaded nanoparticle composition of the present invention.

FIG. 9C, fluorescent microscopic image of the effect of reducing extracellular matrix of the sorafenib-loaded nanoparticle composition of the present invention.

FIG. 10A, the effect of reducing angiogenesis of the sorafenib-loaded nanoparticle composition of the present invention in fibrotic liver.

FIG. 10B, the effect of reducing the diameter of blood vessels of the sorafenib-loaded nanoparticle composition of the present invention in fibrotic liver.

FIG. 10C, fluorescent microscopic image of the effect of reducing the density of blood vessels of the sorafenib-loaded nanoparticle composition of the present invention in fibrotic liver.

### DETAILED DESCRIPTION OF THE PREFERRED EMBODIMENT

The present invention provides a method for ameliorating liver fibrosis in a subject. Firstly, the nanoparticle composition containing tyrosine kinase inhibitor, such as sorafenib, was prepared by utilizing a PEG-PLGA polymer or a PEG-PLGA/PLGA co-polymer. The coating, encapsulation, and in vivo characteristics such as pharmacokinetics and cytotoxicity were also evaluated. Then, the sorafenib-loaded PEG-PLGA nanoparticle composition and the sorafenib-loaded PEG-PLGA/PLGA nanoparticle composition were evaluated regarding their effect to liver cells, and, particularly, regarding their ameliorative effect to fibrotic liver. As shown from the experimental results, the administration of the sorafenib-loaded PEG-PLGA nanopartilce or the sorafenib-loaded PEG-PLGA/PLGA nanoparticle composition of the present invention can reduce the amount of an extracellular matrix, inhibit the activity of hepatic stellate cells, and normalize the blood vessels in fibrotic liver including shrinking of abnormal blood vessels and reducing the microvescular density of fibrotic liver. Hence, the method of the present invention can promote the absorption of tyrosine kinase, such as sorafenib, in specific target tissues, for instance, fibrotic liver tissue, as well as reduce the side-effect thereof. Furthermore, the nanoparticle composition of the present invention can be made into non-conventional dosage form such as injection, which shows great clinical potential for the prevention and treatment of liver fibrosis.

### DEFINITION

As used herein, the terms “fibrosis” and “fibrotic” refer to the pathological changes involving increase of connective tissues in the organs and decrease of actual cells.

As used herein, the term “co-polymer” refers to product of polymeric reactions and includes natural or synthetic heteropolymer, random co-polymer, alternating co-polymer, block co-polymer, graft co-polymer, branch co-polymer, cross-linking co-polymer, trimer, polymeric alloy, and the combination and modification thereof. Polymers can be saturated in true solution, or be saturated or oversaturated in benefit agent, or be suspended as particulates.

### Methods and Materials

### Preparation of the Most Preferable Nanoparticle Composition for Encapsulating Tyrosine Kinase Inhibitor

Firstly, the nanoparticle composition for encapsulating tyrosine kinase inhibitor of the present invention was prepared. Poly (D, L-lactide-co-glycolide), abbreviated as PLGA, is a macromolecular polymeric material with extreme low cytotoxicity and good bioavailability and bio-absorbability. PLGA as well as the co-polymer thereof can decompose into fragments of small molecular weight such as lactic acid and glycolic acid via, for instance, hydrolysis, and can be further metabolized into carbon dioxide and water via Kreb's cycle. Carbon dioxide and water can then be discharged from the human body. The polymer of PEG-PLGA is amphiphilic due to the PLGA proportion and PEG proportion therein being hydrophobic and hydrophilic, respectively. The utilization of PEG-PLGA in nanoparticle composition formation can reduce polydispersity as well as increase stability of the nanoparticle composition in blood circulation.

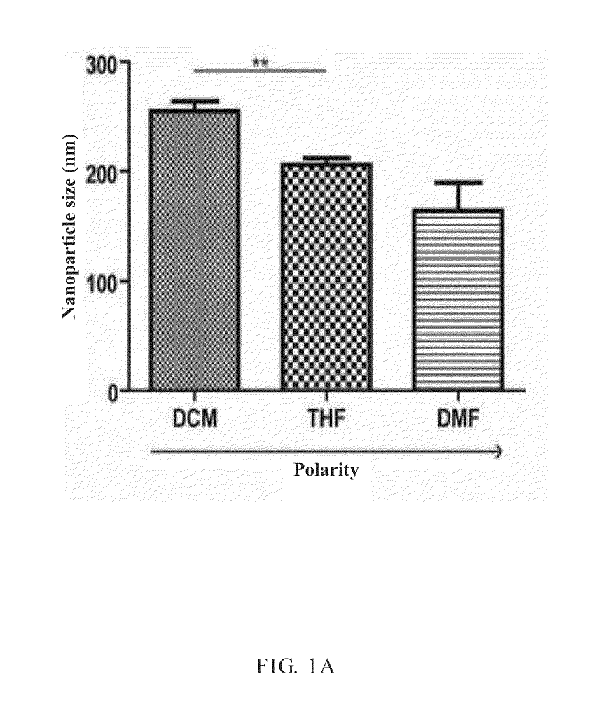

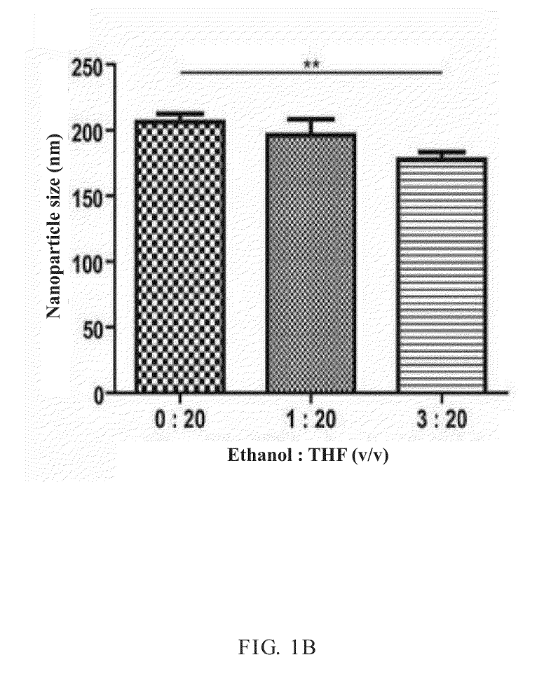

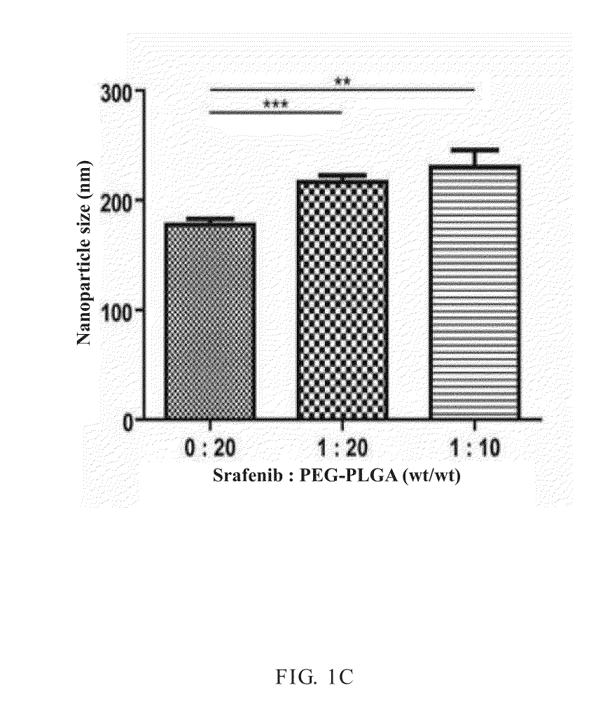

PEG-PLGA and PLGA were purchased from Greensquare Co., Ltd. (Taiwan). PEG-PLGA was placed in solvent of different polarities such as dichloromethane (DCM), tetrahydrofuran (THF), and dimethylformamide (DMF) and the size of the nanoparticle composition formed was measured. As shown in FIG. 1A, the size of the PEG-PLGA nanoparticle composition formed in THF, DCM, and DMF is approximately 200 nm, 250 nm, and 160 nm, respectively, indicating that PEG-PLGA can form nanoparticle compositions having size smaller than 300 nm Such nanoparticle composition size is suitable for in vivo drug delivery. PEG-PLGA was then dissolved in solvent with different ratios of ethanol to THF (v/v). As shown in FIG. 1B, preferably, when the ethanol to THF ratio (v/v) is 3:20, the PEG-PLGA nanoparticle composition formed can be maintained at a size of approximately 200 nm. Furthermore, the PEG-PLGA nanoparticle composition was used to encapsulate sorafenib and the particle size of the sorafenib-loaded nanoparticle composition was measured with different sorafenib to PEG-PLGA ratio (wt/wt). As shown in FIG. 1C, when the sorafenib to PEG-PLGA ratio is 1:20, the size of the sorafenib-loaded PEG-PLGA nanoparticle composition is about 220 nm; when the sorafenib to PEG-PLGA ratio is 1:10, the size of the sorafenib-loaded PEG-PLGA nanoparticle composition is about 240 nm.

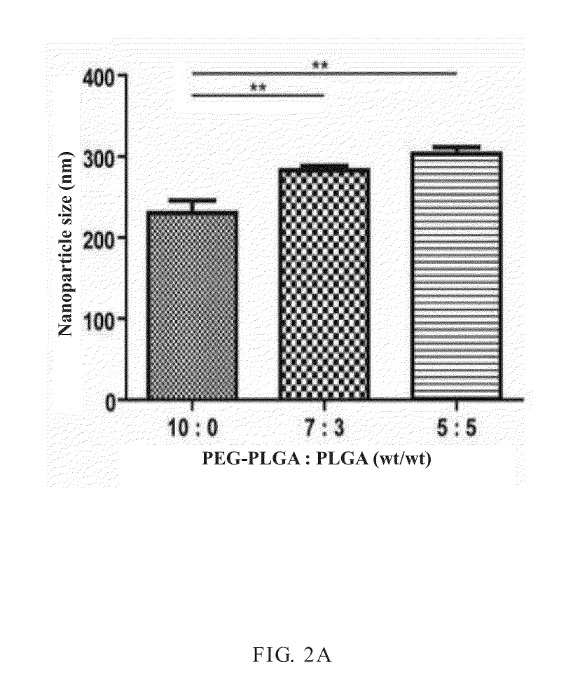

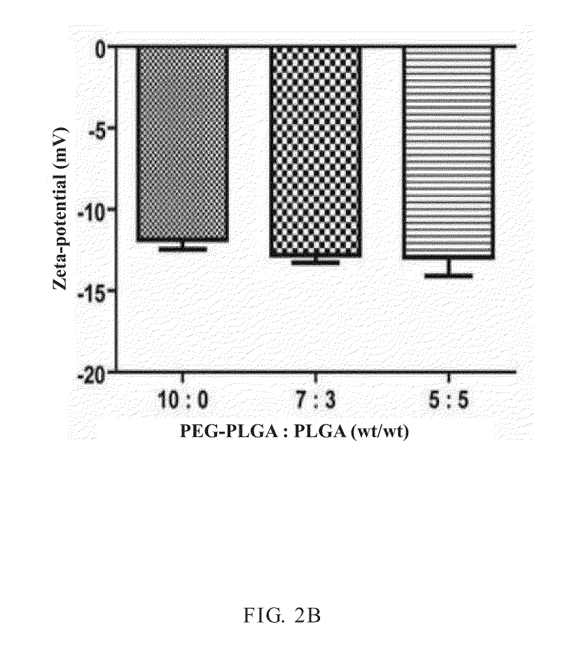

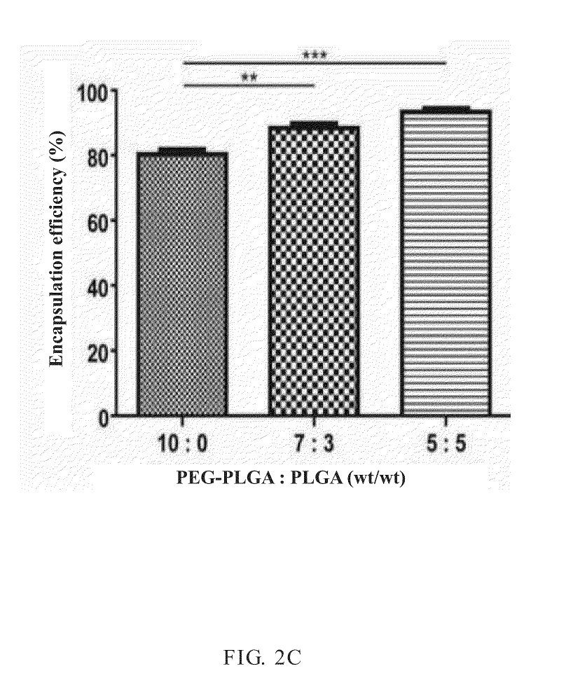

A co-polymer constitutes of PEG-PLGA and PLGA was also provide and evaluated in regard to the ratio (wt/wt) of PEG-PLGA:PLGA therein. Firstly, two types of PEG-PLGA/PLGA co-polymers were made respectively according to the PEG-PLGA:PLGA ratio (wt/wt) of 7:3 and 5:5 and were both dissolved in THE As shown in FIG. 2A, when the PEG-PLGA:PLGA ratio (wt/wt) is 7:3, the size of the nanoparticle composition formed is about 280 nm; when the PEG-PLGA:PLGA ratio (wt/wt) is 5:5, the size of the nanoparticle composition formed is about 300 nm. The zeta-potential of the PEG-PLGA/PLGA co-polymer with different ratios of PEG-PLGA to PLGA was then evaluated, and as shown in FIG. 2B, the results of zeta-potential of the PEG-PLGA/PLGA co-polymers are constant for PEG-PLGA:PLGA ratio of 7:3 or 5:5, indicating similar level of mobility. Furthermore, the encapsulation efficiency of the PEG-PLGA/PLGA co-polymer with different ratios of PEG-PLGA to PLGA was also evaluated, and as shown in FIG. 2C, when the PEG-PLGA:PLGA ratio (wt/wt) is 5:5, the nanoparticle composition formed exhibits optimal encapsulation efficiency of about 95%.

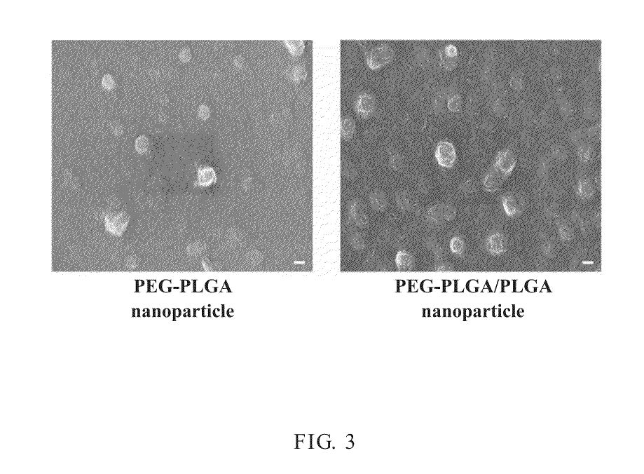

It is therefore known from above that nanoparticle composition formed by 50% PEG-PLGA and 50% PLGA possesses the best encapsulation efficiency; hence, PEG-PLGA/PLGA co-polymer constitute of 50% PEG-PLGA and 50% PLGA as well as polymer of 100% PEG-PLGA were selected to encapsulate sorafenib to give sorafenib-loaded nanoparticle compositions for the embodiments hereinafter. In addition, characteristics of such nanoparticle compositions formed were analyzed. As shown in Table 1, the average diameter and the polydispersity index (PDI) of the sorafenib-loaded nanoparticle composition formed using polymer of 100% PEG-PLGA are 230.1±15.6 nm and 0.352±0.024, respectively; whereas the average diameter and the PDI of the sorafenib-loaded nanoparticle composition formed using co-polymer constitute of 50% PEG-PLGA and 50% PLGA are 303.3±7.8 nm and 0.214±0.014, respectively. The electron microscopic images of the sorafenib-loaded nanoparticle compositions are illustrated in FIG. 3 and the scale represents 100 nm

TABLE 1

| | PEG-PLGA nanoparticle | PEG-PLGA/PLGA |
|---|---|---|
| | composition | nanoparticle composition |
| Average diameter | 230.1 ± 15.6 | 303.3 ± 7.8 |
| (nm) | | |
| Polydispersity index | 0.352 ± 0.024 | 0.214 ± 0.014 |
| (PDI) | | |

### Example 1

Drug release rate of the nanoparticle composition containing tyrosine kinase inhibitor

0.1 mg of the sorafenib-loaded nanoparticle compositions were placed in microcentrifuge tubes and were dispersed in 1 mL of phosphate buffered saline (PBS). The microcentrifuge tubes were then placed in Shaking Incubator at 37° C. After incubation for a predetermined period of time, the sediment was dissolved in dimethyl sulfoxide (DMSO) and was analyzed using a spectrophotometer at a wavelength of 270 nm.

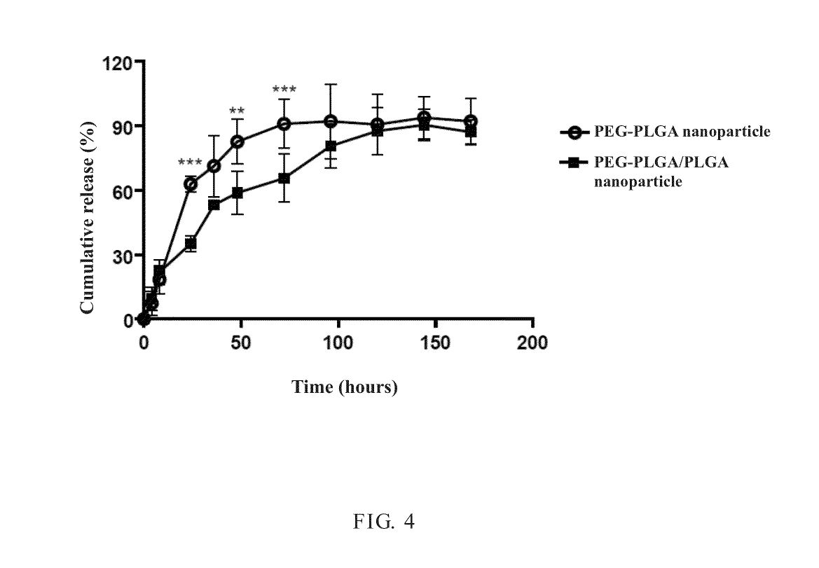

As shown in FIG. 4, the cumulative release rate of the sorafenib-loaded PEG-PLGA nanoparticle composition and the sorafenib-loaded PEG-PLGA/PLGA nanoparticle composition within 168 hours, the sorafenib-loaded PEG-PLGA nanoparticle composition exhibits higher release rate within 72 hours and reaches cumulative release of about 100% at the 72^{nd} hour; on the other hand, the sorafenib-loaded PEG-PLGA/PLGA nanoparticle composition shows slower and steadier release rate and reaches cumulative release of 100% at the 120^{th} hour.

### Example 2

### Cytotoxicity of the Nanoparticle Composition Containing Tyrosine Kinase Inhibitor

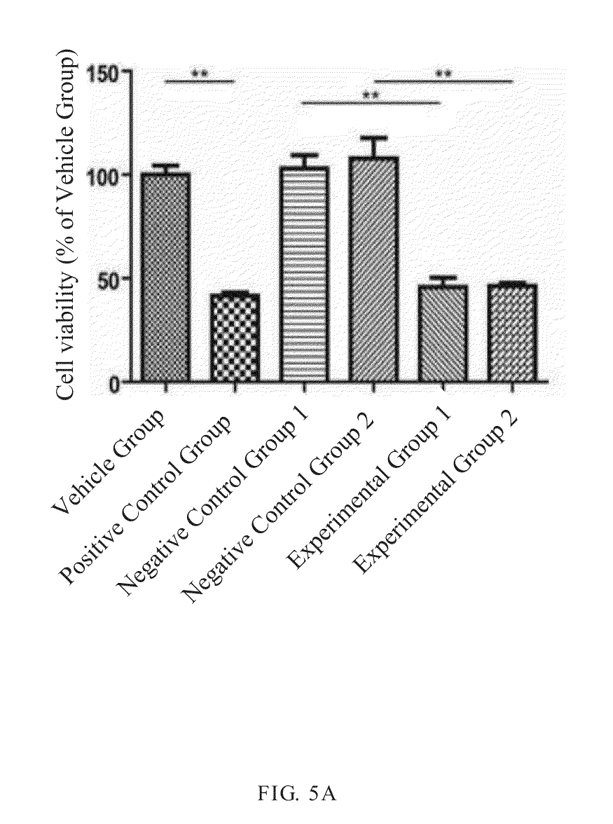

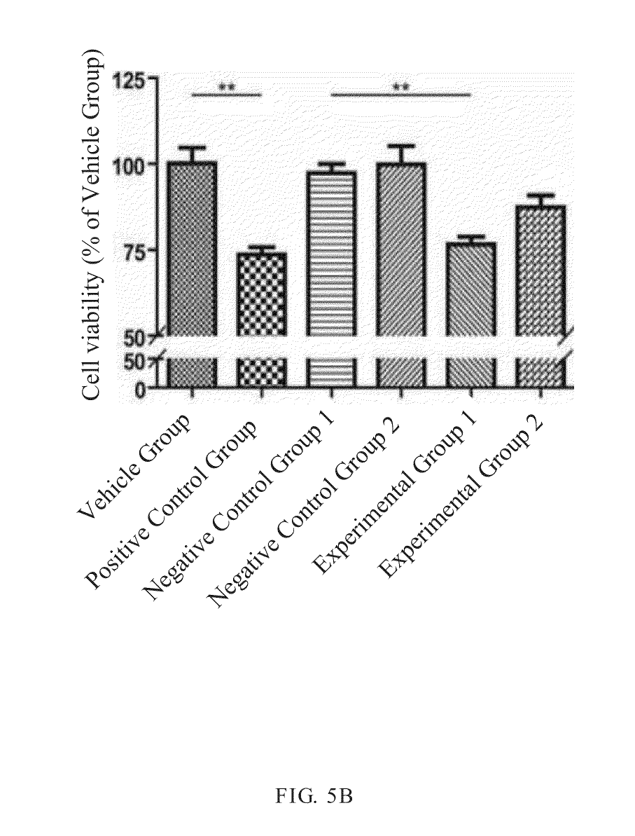

To evaluate the cytotoxicity of the sorafenib-loaded PEG-PLGA nanoparticle composition and the sorafenib-loaded PEG-PLGA/PLGA nanoparticle composition of the present invention, a human umbilical vein endothelial cell (HUVEC) was provided to represent the target cell with angiogenesis, while a hepatic stellate cell (HSC) was provided to represent the fibrotic target cell. The HSC was purchased from ScienCell Research Laboratories (#5300, San Diego, Calif., USA) and the HUVEC was obtained from Bioresource Collection and Research Center, Food Industry Research and Development Institute (BCRC number: H-UV001, Hsinchu, Taiwan). Firstly, HUVECs and HSCs were planted in 96-well plates at a density of 2,000 cells/well and were left overnight for adhesion. Then, different forms of sorafenib or drug carriers were added. After 48 or 72 hours, 3-(4, 5-cimethylthiazol-2-yl)-2, 5-diphenyl tetrazolium bromide (MTT), which dissolved in PBS, was added to each well and the cells were further incubated at 37° C. for 3 hours. Finally, to each well, 50 μL of DMSO were added and a spectrophotometer was used to analyze the result at a wavelength of 570 nm. Cells treated with sorafenib-loaded PEG-PLGA particle and sorafenib-loaded PEG-PLGA/PLGA nanoparticle composition served as Experimental Group 1 and Experimental Group 2, respectfully. Cells treated with PEG-PLGA particle alone and PEG-PLGA/PLGA nanoparticle composition alone, on the other hand, served as Negative Control Group 1 and Negative Control Group 2, respectfully. In addition, cells without any treatment served as Vehicle Group, while cells treated with free sorafenib served as Positive Control Group. As shown in FIG. 5A, for HUVECs, the cell viability is below 50% when treated with free sorafenib, the sorafenib-loaded PEG-PLGA nanoparticle composition (Experimental Group 1), and the sorafenib-loaded PEG-PLGA/PLGA nanoparticle composition (Experimental Group 2), indicating that both the sorafenib-loaded PEG-PLGA nanoparticle composition and the sorafenib-loaded PEG-PLGA/PLGA nanoparticle composition of the present invention can release sorafenib in target cells. Meanwhile, according to the results of cell viability of the cells treated with the PEG-PLGA particle alone (Negative Control Group 1) and PEG-PLGA/PLGA nanoparticle composition alone (Negative Control Group 2), it is confirmed that the PEG-PLGA and PEG-PLGA/PLGA co-polymer are non-cytotoxic and will not result in death of the target cells.

For HSCs, as shown in FIG. 5B, the cell viability of the cells treated with free sorafenib is about 75%, whereas the cell viability of the cells treated with sorafenib-loaded PEG-PLGA nanoparticle composition (Experimental Group 1) and sorafenib-loaded PEG-PLGA/PLGA nanoparticle composition (Experimental Group 2) are 75% and 88%, respectively, indicating that both the sorafenib-loaded PEG-PLGA nanoparticle composition and the sorafenib-loaded PEG-PLGA/PLGA nanoparticle composition of the present invention can release sorafenib in target cells and reduce cell viability. On the other hand, according to the results of cell viability of the cells treated with the PEG-PLGA particle alone (Negative Control Group 1) and PEG-PLGA/PLGA nanoparticle composition alone (Negative Control Group 2), it is confirmed that the PEG-PLGA and PEG-PLGA/PLGA co-polymer are non-cytotoxic and will not result in death of the target cells.

### Example 3

### Pharmacokinetics of Injected Coumarin 6 (C6)-Loaded Nanoparticle Composition

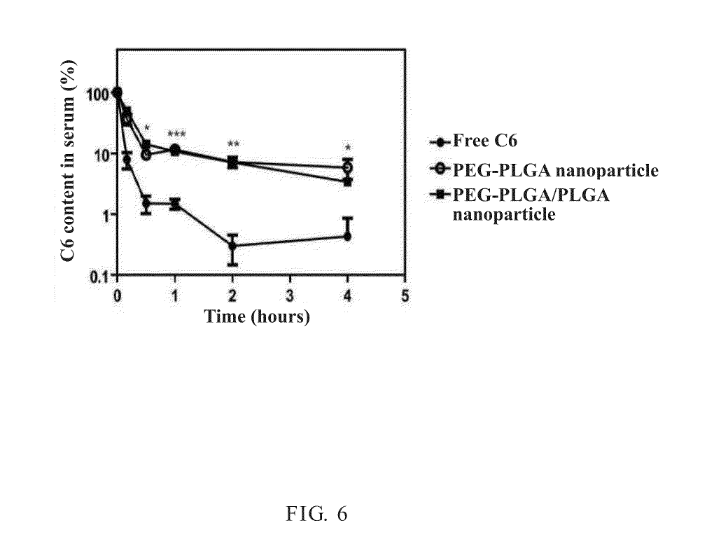

As shown in FIG. 6, after injection of C6-containing serum and the C6-loaded PEG-PLGA/PLGA nanoparticle composition of the present invention for 0.5 to 1 hour, the C6 concentrations in blood thereof are approximately 10%, whereas the C6 concentration in blood treated with C6 alone is about 1%, indicating that the PEG-PLGA nanoparticle composition and PEG-PLGA/PLGA nanoparticle composition of the present invention can effectively reduce the release rate of drug in blood and result in more preferable pharmacokinetic. On the other hand, after injection of C6-loaded PEG-PLGA nanoparticle composition and C6-loaded PEG-PLGA/PLGA nanoparticle composition of the present invention for 1 to 4 hours, the C6 concentration in blood gradually stabilized, indicating that the PEG-PLGA nanoparticle composition and PEG-PLGA nanoparticle composition of the present invention lead to a steady release of drug in blood in the long term.

### Example 4

### Absorption of C6-Loaded Nanoparticle Composition in Diseased Liver

The C6-loaded nanoparticle composition was prepared according to the same emulsification method as the preparation of the sorafenib-loaded PEG-PLGA nanoparticle composition. C6 was injected to C3H mice with or without the nanoparticle encapsulation at a dosage of 0.1 mg/kg via tail intravenous injection. 40 μL of blood from the tail artery of the mice were collected and mixed with ethylenediaminetetraacetic acid (EDTA) at different period of time. Fluorescence intensity was measured under an excitation wavelength of 485 nm and an emission wavelength of 538 nm. Standard curves of the C6 or C6-loaded PLGA nanoparticle composition in blood were used to calculate the content of C6 in mice.

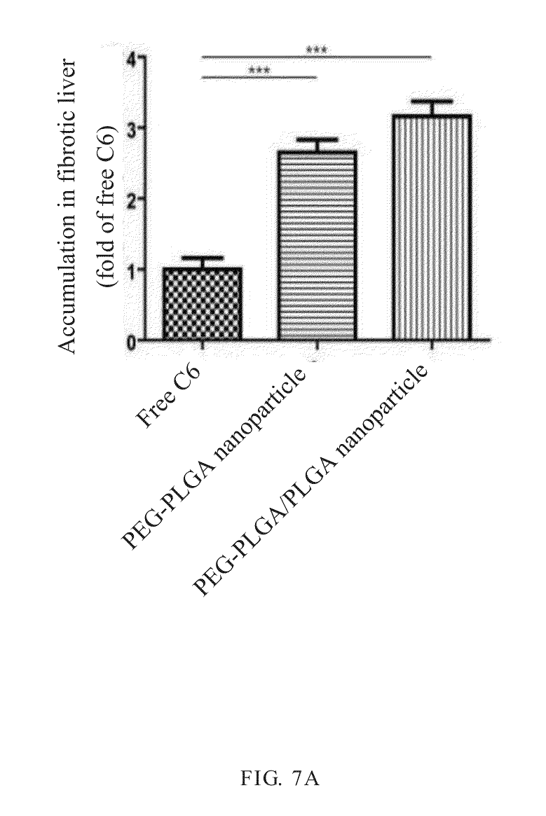

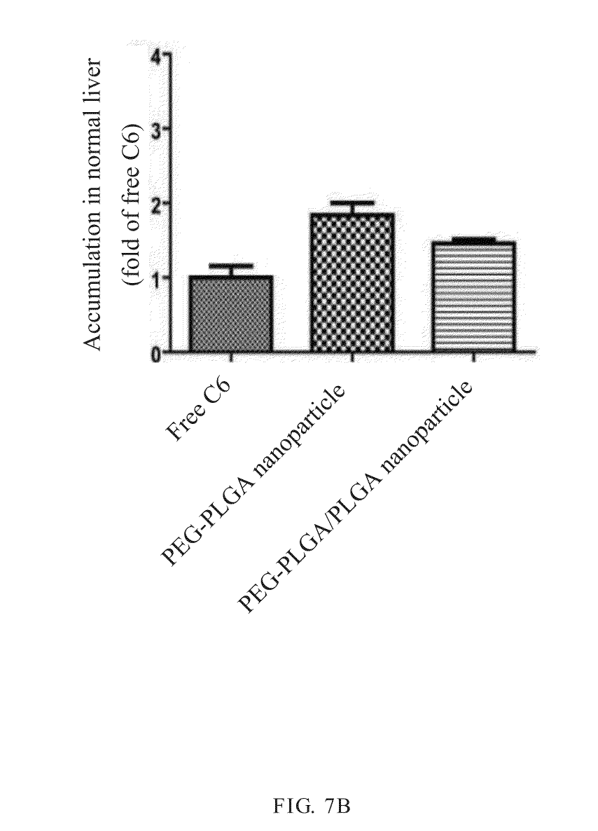

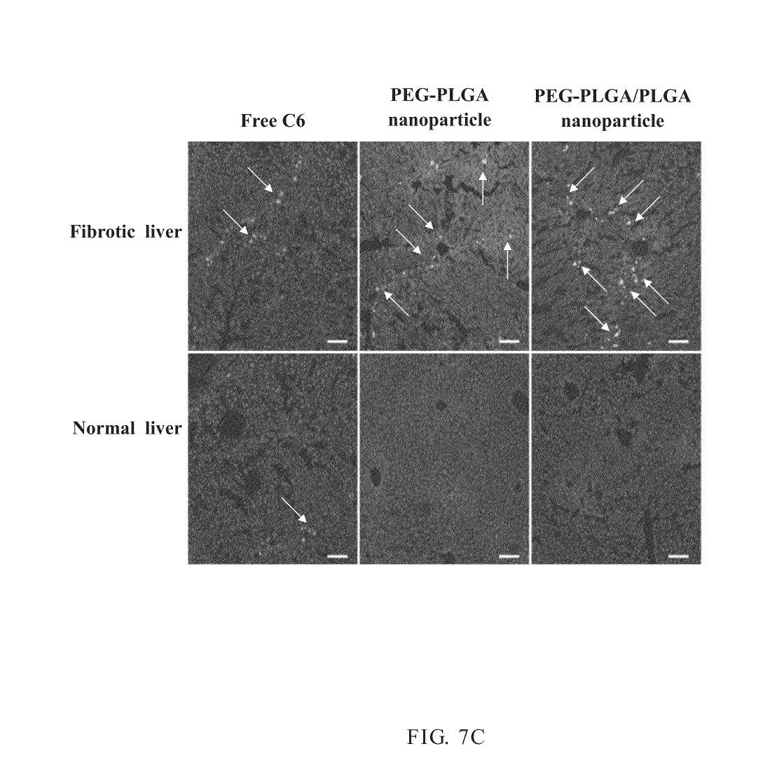

As shown in FIG. 7A, in the fibrotic liver, the accumulation of C6-loaded PEG-PLGA nanoparticle composition is about 2.7-fold greater than that of the Control Group, while the accumulation of C6-loaded PEG-PLGA nanoparticle composition is about 3-fold greater than that of the Control Group. In comparison to FIG. 7B, neither the C6-loaded PEG-PLGA nanoparticle composition nor the C6-loaded PEG-PLGA/PLGA nanoparticle composition would significantly accumulate in normal liver. As further illustrated in FIG. 7C, for fibrotic liver cell, both the amount of C6 (white arrows) of the PEG-PLGA nanoparticle composition and PEG-PLGA/PLGA nanoparticle composition are increased, indicating that when coated with the PEG-PLGA nanoparticle composition or PEG-PLGA/PLGA nanoparticle composition of the present invention, the accumulation of C6 in the target cell (ie. fibrotic liver cell) is enhanced. Such accumulation of C6 is not seen in normal cells. It is therefore shown that the PEG-PLGA nanoparticle composition and PEG-PLGA/PLGA nanoparticle composition of the present invention can promote the substances contained therein to accurately accumulate in target cell (ie. fibrotic liver cell).

### Example 5

### Effect of Ameliorating Liver Fibrosis of the Tyrosine Kinase Inhibitor-Loaded Nanoparticle Composition

Carbon tetrachloride (CCl₄) was used to induce liver fibrosis in mice. 100 μL of 16% (v/v) CCl₄ were added to olive oil and were blended into fodder. 4-week-old mice were fed with such fodder for 8 weeks (until the mice were 12 weeks old). At the fifth week (ie. The mice were 9 weeks old), the sorafenib-loaded PEG-PLGA nanoparticle composition and the sorafenib-loaded PEG-PLGA/PLGA nanoparticle composition were administered at a dosage of 10 mg/kg via tail intravenous injection. The mice were subjected to such injection twice a week for four weeks. CCl₄ induction was consistent while the sorafenib administration took place. The CCl₄ induced fibrotic liver mice were used the Vehicle Group, while the mice treated with free sorafenib were used as the Positive Control Group. The mice treated with the sorafenib-loaded PEG-PLGA nanoparticle composition and the sorafenib-loaded PEG-PLGA/PLGA nanoparticle composition were used as Experimental Group 1 and Experimental Group 2, respectively. The mice treated without sorafenib but with the PEG-PLGA nanoparticle composition and the PEG-PLGA/PLGA nanoparticle composition were used as Negative Control Group 1 and Negative Control Group 2, respectively.

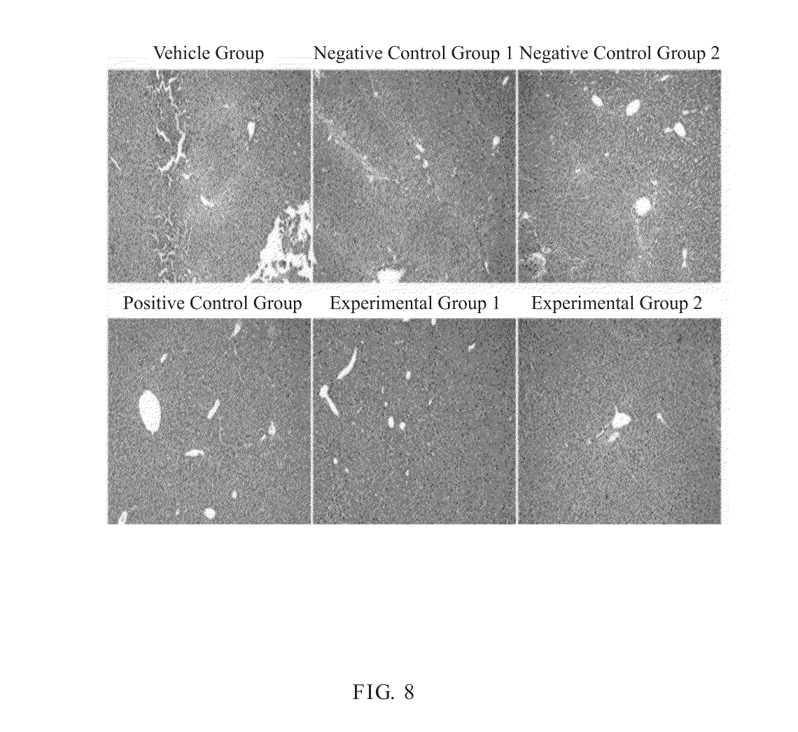

As shown in FIG. 8, the Vehicle Control Group exhibits severe fibrosis (white region). Due to lack of sorafenib, the fibrosis does not significantly ameliorated for the mice treated with Negative Control Group 1 and Negative Control 2. As for the mice treated with Experimental Group 1 and Experimental Group 2, when comparing to the mice treated with the Vehicle Control group, notable reduction of the amount and scope of fibrosis (white region) can be observed, indicating that the sorafenib-loaded PEG-PLGA nanoparticle composition and the sorafenib-loaded PEG-PLGA/PLGA nanoparticle composition can effectively suppress liver fibrosis. Moreover, when comparing to the mice treated with free sorafenib alone (the Positive Control Group), the sorafenib-loaded PEG-PLGA nanoparticle composition and the sorafenib-loaded PEG-PLGA/PLGA nanoparticle composition of the present invention clearly exhibit better effect regarding suppression of fibrosis, indicating that the use of the PEG-PLGA nanoparticle composition and PEG-PLGA/PLGA nanoparticle composition of the present invention can enhance the ameliorating effect of sorafenib for the treatment of liver fibrosis.

### Example 6

### Reduction of Extracellular Matrix and Activation of HSCs by the Tyrosine Kinase Inhibitor-Loaded Nanoparticle Composition

The expression of α-smooth muscle actin (α-SMA) is an indicator for HSC activation. The present invention evaluates the effect of the sorafenib-loaded PEG-PLGA nanoparticle composition and the sorafenib-loaded PEG-PLGA/PLGA nanoparticle composition in HSC activation.

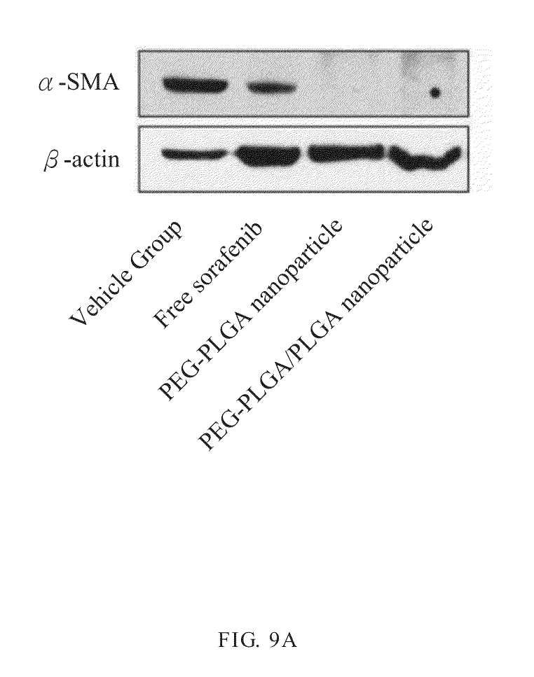

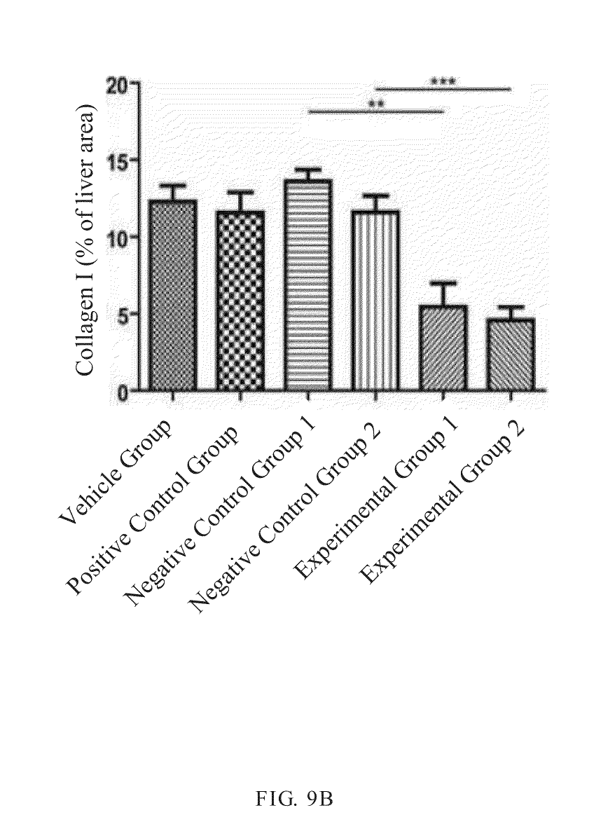

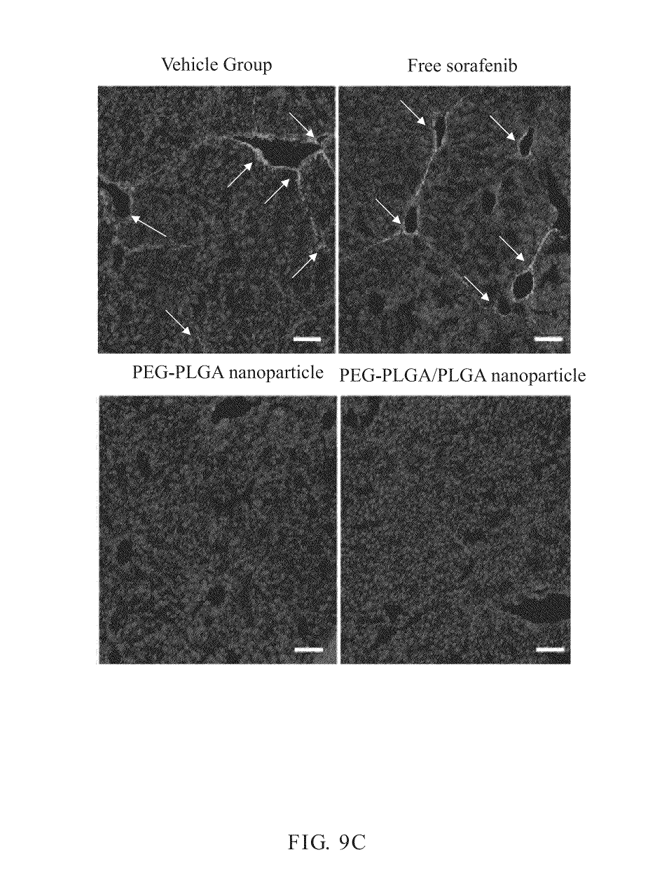

As shown in FIG. 9A, the western blotting reveals that α-SMA expression is suppressed when the mice were treated with free sorafenib, indicating inhibition of HSC activation. On the other hand, specifically, when the mice were treated with the sorafenib-loaded PEG-PLGA nanoparticle composition of the present invention, no α-SMA expression is seen, indicating that the use of the PEG-PLGA nanoparticle composition of the present invention can result in optimal effect of sorafenib in terms of HSCs activation suppression.

Collagen is the most abundant protein in not only the human body but also the extracellular matrix. The present invention examines the content collagen I to evaluate the effect of the sorafenib-loaded PEG-PLGA nanoparticle composition and the sorafenib-loaded PEG-PLGA/PLGA nanoparticle composition of the present invention. The mice without any treatment served as the Vehicle Group, while the mice treated with free sorafenib served as the Positive Control Group. The mice treated with the sorafenib-loaded PEG-PLGA nanoparticle composition and the sorafenib-loaded PEG-PLGA/PLGA nanoparticle composition served as Experimental Group 1 and Experimental Group 2, respectively. The mice treated without sorafenib but with the PEG-PLGA nanoparticle composition and PEG-PLGA/PLGA nanoparticle composition alone served as Negative Control Group 1 and Negative Control Group 2, respectively.

As shown in FIG. 9B, the content of extracellular matrix is significant reduced to less than 5% of the area of the liver when treated with the Experimental Group 1 and the Experimental Group 2. The effects of reduction of extracellular matrix of both Experimental Group 1 and 2 are about 3-fold better than that of the Positive Control Group, indicating that the use of the PEG-PLGA nanoparticle composition and the PEG-PLGA/PLGA nanoparticle composition of the present invention can effectively promote the effect of sorafenib in terms of extracelluar matrix reduction. Furthermore, as shown in FIG. 9C, in which the illuminated regions (specified as white arrows) indicate extracellular matrix, the reduction of extracellular matrix is not noteworthy when mice were treated with free sorafenib alone. However, when mice were treated with the sorafenib-loaded PEG-PLGA nanoparticle composition and the sorafenib-loaded PEG-PLGA/PLGA nanoparticle composition of the present invention, almost no illuminated regions can be observed, indicating that the sorafenib-loaded PEG-PLGA nanoparticle composition and the sorafenib-loaded PEG-PLGA/PLGA nanoparticle composition can significantly reduce the amount of extracellular matrix.

### Example 7

### Reduction of Angiogenesis and Abnormal Blood Vessels by the Tyrosine Kinase Inhibitor-Loaded Nanoparticle Composition

A glycoprotein known as von Willebrand factor (vWF) is related to coagulation and is used as a cellular marker for angiogenesis in tumor. This embodiment evaluates the effect of angiogenesis by the sorafenib-loaded PEG-PLGA nanoparticle composition and the sorafenib-loaded PEG-PLGA/PLGA nanoparticle composition of the present invention.

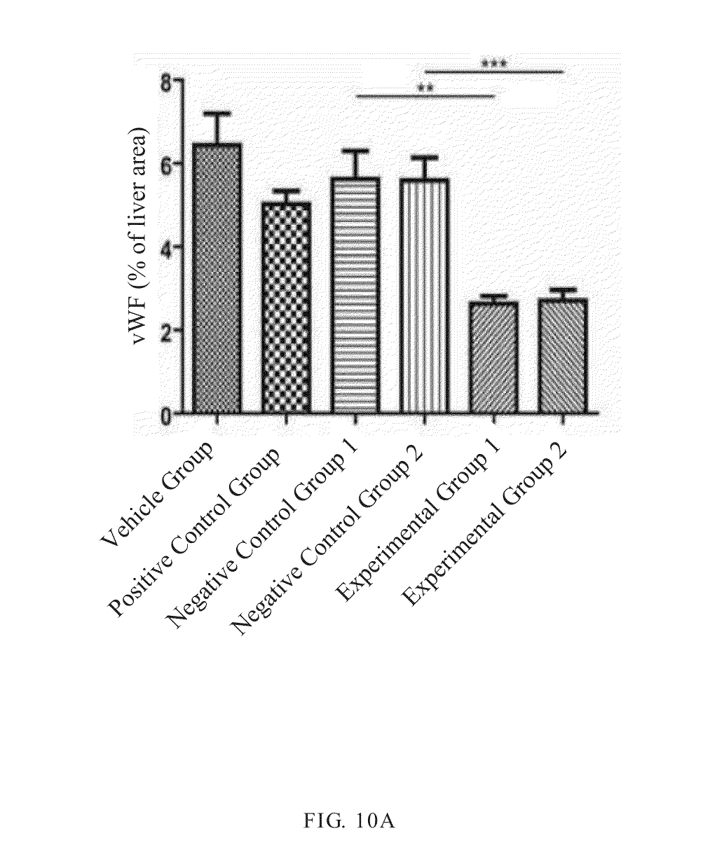

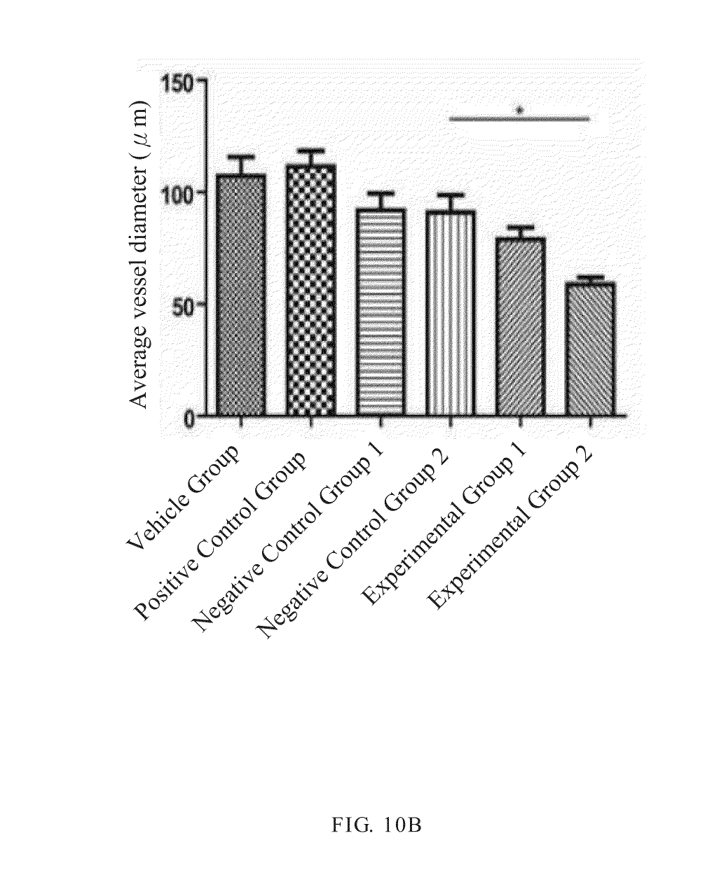

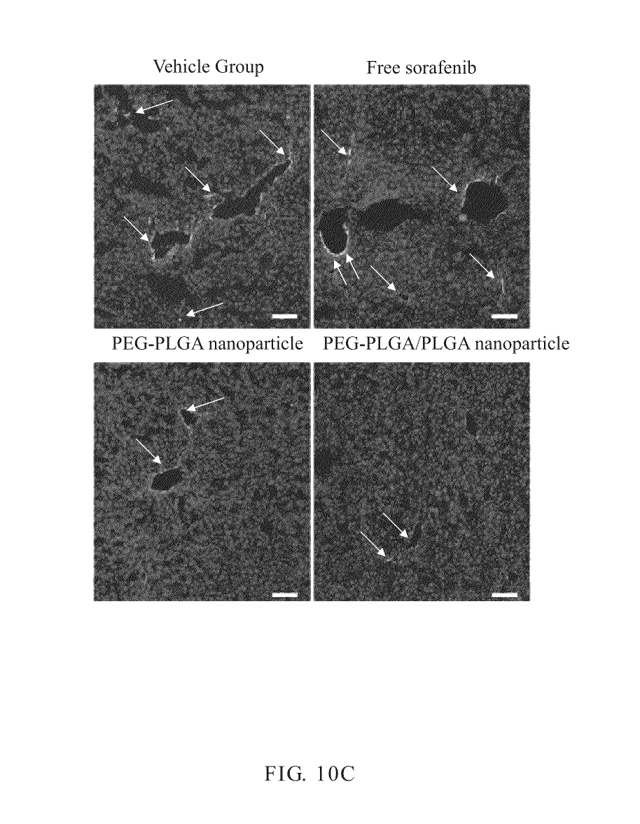

As shown in FIG. 10A, angiogenesis is significantly reduced, to about 2-fold, when mice were treated with both the sorafenib-loaded PEG-PLGA nanoparticle composition (Experimental Group 1) and the sorafenib-loaded PEG-PLGA/PLGA nanoparticle composition (Experimental Group 2) comparing to the mice treated with free sorafenib alone (Positive Control Group) and the mice without any treatment (Vehicle Group), indicating that the use of the PEG-PLGA nanoparticle composition and the PEG-PLGA/PLGA nanoparticle composition of the present invention can promote the effect of sorafenib in terms of angiogenesis reduction.

Moreover, this embodiment also evaluates the effect of the sorafenib-loaded PEG-PLGA nanoparticle composition and the sorafenib-loaded PEG-PLGA/PLGA nanoparticle composition of the present invention to abnormal blood vessels, and particularly, the diameter thereof. As shown in FIG. 10B, the average diameter of the blood vessel of the mice without any treatment (Vehicle Group) is about 110 μm. When the mice were treated with the sorafenib-loaded PEG-PLGA nanoparticle composition and the sorafenib-loaded PEG-PLGA/PLGA nanoparticle composition, the average diameter of the blood vessels of the mice significantly reduced, and, in particular, when the mice were treated with the sorafenib-loaded PEG-PLGA/PLGA nanoparticle composition, the average diameter of the blood vessels of the cells is reduced to about 60 μm. In addition, as shown in FIG. 10C, when the mice were treated with the sorafenib-loaded PEG/PLGA nanoparticle composition or the sorafenib-loaded PEG-PLGA/PLGA nanoparticle composition of the present invention, the microvescular density thereof (indicate as white arrows) is significantly lower than the mice without any treatment or treated with free sorafenib alone, indicating that the sorafenib-loaded PEG/PLGA nanoparticle composition and the sorafenib-loaded PEG-PLGA/PLGA nanoparticle composition of the present invention can effectively reduce the diameter and density of capillary in fibrotic liver tissue and promote microvascular normalization.

In summary, the PEG-PLGA polymer of the present invention having low polydispersity can form sorafenib-loaded nanoparticle composition with good hydrophilic and hydrophobic properties which exhibit good stability in blood circulation. The increase of the PLGA content in the PEG-PLGA/PLGA co-polymer of the present invention can enhance the size and the drug encapsulation as well as delay the drug release of the nanoparticle composition formed thereof. The sorafenib-loaded PEG/PLGA or the sorafenib-loaded PEG-PLGA/PLGA nanoparticle composition can be prepared as injection for administration which prolong the existence of sorafenib in blood circulation, promote the absorption of sorafenib in fibrotic liver, reduce the cytotoxicity of sorafenib, and enhance the bioavailability of sorafenib. Hence, the administration of sorafenib-loaded PEG-PLGA nanoparticle composition and the sorafenib-loaded PEG-PLGA/PLGA nanoparticle composition of the present invention can not only effectively ameliorate liver fibrosis but also shrink the abnormal angiogenesis in fibrotic liver, which, then result in reduction of microvascular density and normalization of blood vessel in fibrotic liver.

The method for ameliorating liver fibrosis in a subject according to the present invention is applicable and valuable to the industry. Those embodiments above are better results, and should not, however, be considered to limit the scope of the invention. It is contemplated that modifications will readily occur to those skilled in the art, which modifications will be within the spirit of the invention and scope of the appended claims.

What is claimed is:

1. A method for ameliorating liver fibrosis in a subject, comprising administering to the subject a nanoparticle composition comprising a tyrosine kinase inhibitor, wherein the nanoparticle composition enhances the absorption of the tyrosine kinase inhibitor in fibrotic liver.

2. The method of claim 1, wherein the nanoparticle composition is a polymer of poly(ethylene glycol-b-(D, L-lactide-co-glycolide) (PEG-PLGA), or a co-polymer of poly(ethylene glycol-b-(D,L-lactide-co-glycolide)/poly (D, L-lactide-co-glycolide (PEG-PLGA/PLGA).

3. The method of claim 2, wherein the content of PEG-PLGA in the co-polymer of PEG-PLGAA/PLGA is from 50% to 70% by weight.

4. The method of claim 2, wherein the content of PEG-PLGA and the content of PLGA in the co-polymer of PEG-PLGA/PLGA are both 50% by weight.

5. The method of claim 1, wherein the nanoparticle composition is in an injectable form.

6. The method of claim 1, wherein the tyrosine kinase inhibitor is Sorafenib.

7. The method of claim 1, wherein the nanoparticle composition delays the release of the tyrosine kinase inhibitor in blood circulation.

8. The method of claim 1, wherein the nanoparticle composition reduces the accumulation of an extracellular matrix in fibrotic liver.

9. The method of claim 8, wherein the extracellular matrix is collagen.

10. The method of claim 1, wherein the nanoparticle composition inhibits the activity of hepatic stellate cells.

11. The method of claim 1, wherein the nanoparticle composition reduces the angiogenesis in fibrotic liver.

12. The method of claim 11, wherein the nanoparticle composition further reduces the density and diameter of newly developed blood vessels during the angiogenesis in fibrotic liver.
